(19) United States
(12) Patent Application Publication
(10) Pub. No.: US 2023/0226068 A1
(43) Pub. Date: Jul. 20, 2023

(54) METHOD, SYSTEM, FORMULATION AND KIT FOR THE TREATMENT OF ONYCHOMYCOSIS

(71) Applicant: GEORG SCHUELE, PORTOLA VALLEY, CA (US)

(72) Inventor: GEORG SCHUELE, PORTOLA VALLEY, CA (US)

(21) Appl. No.: 17/567,081

(22) Filed: Dec. 31, 2021

(51) Int. Cl. A61K 31/525; A61K 45/06; A61P 31/00

(52) U.S. Cl. CPC A61K 31/525; A61K 45/06; A61P 31/00

(57) ABSTRACT

A kit for treating an infection of a nail includes: a formulation including urea, and a photoactive dye to stain the nail; and a device for illuminating the stained nail The device includes: an illuminator for emitting light to the stained nail having the formulation applied thereto; drive and control electronics for driving and controlling the illuminator; an initiator for commencing illumination of the stained nail having the formulation applied thereto; and a power source for supplying power to the drive and control electronics and the illuminator. An associated method of treating the nail includes applying the formulation to the nail, and illuminating the stained nail having the formulation applied thereto with light emitted from the device.

### BACKGROUND OF THE INVENTION

### Field of the Invention

This invention relates to the treatment of onychomycosis, and in particular, it relates to a novel method, a handheld compact photo activation system, and a novel formulation for the photodynamic treatment of onychomycosis. The system, method and formulation are used to allow an effective photodynamic treatment of the infected toe nail utilizing photoactivation of the dye. Optimal treatment outcomes are driven by an effective penetration of the nail bed by the photoactive agent and a compact illumination device for ease of use. In an embodiment the photoactive formulation includes Urea which greatly enhances the transungual penetration. A beneficial formulation uses Methylene Blue (MB), Riboflavin-5-phosphate (FMH) or Riboflavin (RF) as a photoactive agent. In a beneficial embodiment the method and system contains of a battery driven compact handheld LED light source.

### Description of Related Art

Onychomycosis, or toe nail fungus is an infectious disease of the nail. The patient population is huge as about 10% of global population is affected. Worldwide overall about 780 million patients are affected in 2020. There also is a huge shift in market dynamics from 92% of oral treatments in 2009 to now 55% topical treatments in 2019 with a clear trend toward more topical solutions.

Existing treatment options for oral treatments are driven by different drugs like Terbinafine, Itraconazole, Fluconazole. They have a high mycological cure rate of 50-70% and moderate total cure rate of 14-38% but come with a considerable severe side effect of liver enzyme abnormalities, headaches, as well as gastrointestinal problems or rashes. Especially the significant possible liver side effects drive the now high adoption towards the less efficacious topical treatment solutions.

Topical drugs include Efinaconazole, Tavaborole, Ciclopirox and Amorolfine which allow some treatment success of 29-50% mycological cure rate and 5-18% total cure rate. But at the same time the topical treatment options come with a significant favorable safety profile only limited to local skin irritations. The lower efficacy is compensated with an improved side effect profile.

Typically, topical treatment is indicated for nails with relatively low fungus infestation and a low Onychomycosis Severity Index (OSI) of less than 15, while deep nail infestation is treated with oral treatment options for OSI > 15 with close to full nail involvement.

Other treatment options have been clinically tested which include Intense Pulsed Light (IPL), thermal laser treatment, plasma treatment or Low-Level Light Therapy (LLT). Although these modalities show a treatment effect, the efficacy is low compared to the available topical and oral pharmacological solutions. The FDA even had to introduce a low-end clinical endpoint which only relates to a clearing of the nail appearance - a visual improvement - which does not represent a mycological cure or full cure which are associated with the pharmacological solutions.

Growth rates of the nails are relatively low with 3.5 mm/month for finger nails and 1.6 mm/month for toe nails. This means that for a full regrowth cycle of the finger nail takes about 3-4 months while toe nails typically need up to a full year to fully regrow. That’s also why all treatment regimens require daily treatments for a full year for topical treatments. For oral treatment the regimens prescribe oral medication every week for a year. The typical treatment cost for full treatment cycles can be significant, with multiple thousands of dollars spent just on treatment supplies and drugs. But this clearly also includes a significant treatment burden on the patient as daily compliance with a treatment regimen is highly unlikely to happen in real world scenarios.

Photodynamic therapy (PDT) has been fully approved for use in many different clinical applications. In dermatology it is approved for the treatment of actinic keratoses using aminolevulinic acid (ALA). In ophthalmology it’s approved for the treatment of choridal neovascularizations in wet Agerelated Macula Degeneration (wet AMD) using Verteporfin, a benzoporphyrin derivative. Also corneal crosslinking for the treatment of keratoconus using Riboflavin is fully approved and represents the current standard of care.

PDT for the treatment of onychomycosis has been clinically tested utilizing many different photoactive drugs. These include MB, RF, Toluidine Blue, ALA as well as methyl aminolevulinate (MAL). Although initial results are encouraging, the overall treatment success was limited by several different issues. For all photoactive drugs there is no diffusion of the drug into the nail itself. This achieves only a superficial treatment effect while the core problem of deep nail plate infections is maintained and not treated. Also, the availability of appropriate light sources is limited to large LED panels as used for the treatment of large skin diseases or expensive high powered diode laser. This also excludes the possible treatment of the patient at home and thus requires full attention and treatment of either the podiatrist or dermatologist in possession of these light sources.

What is needed are new methods, systems, formulations and kits to advance the standard of care and enable a new and efficient treatment of onychomycosis and other nail infections.

### SUMMARY OF THE INVENTION

Accordingly, the present invention is directed to a method of treating infections of the nail using photodynamic therapy that substantially obviates one or more of the problems due to limitations and disadvantages of the related art.

One object of the present invention is to provide efficient photodynamic treatment of the nail due to enhanced diffusion of the photoactive dye into the nail bed, allowing treatment of spores and bacteria which are deeply embedded in the nail plate, nail bed or root of the nail. The enhanced dye diffusion comprises of a formulation which contains Urea to soften the nail during the staining process.

It is a further object of the current invention to provide a compact battery driven light emitting system that enables activation of said photoactive dye by emitting a spectrum of wavelengths which significantly overlaps the absorption spectrum of the dye. This system also provides sufficient brightness to enable efficient treatment. The system is configured to be used by the patient at home.

In one aspect, a method of treating a nail infection includes staining the nail plate with a photoactive dye in a formulation containing urea, allowing the dye to diffuse deep into the nail plate, illuminating the nail with a spectrally matched light source to activate the photoactive dye at light irradiance and exposure levels sufficient to treat the nail. The system utilized for the illumination contains a battery to power the system and includes a timer to have control means for the exposure duration.

In yet another aspect, a method of treating nail infection includes a treatment regimen that gets repeated multiple times. In some embodiments the treatment cycle is repeated a fixed number of times, while in other embodiments the number of treatment cycles depends on the evaluation of the treated nail. In yet another aspect a kit or other product is provided for treating a nail having an infection. The kit includes: a formulation containing urea and a photoactive dye to stain the nail; and a system for illuminating the stained nail, wherein the system includes: an illuminator for emitting light to the stained nail having the formulation applied thereto; drive and control electronics for driving and controlling the illuminator; an initiator for commencing illumination of the stained nail having the formulation applied thereto; and a power source for supplying power to the drive and control electronics and the illuminator.

In some embodiments, the formulation may be provided, for example, in a tube, in a jar, in a roll-on applicator, embedded in one or more applicator strips, or in a prefilled brush pen. These are but a few examples and are non-limiting. In some embodiments, the illuminator includes an array of light sources. In some embodiments, the light sources are light emitting diodes (LEDs). In some embodiments, the photoactive dye has an activation or absorption spectrum, and the emitted light of the illuminator is spectrally matched to the activation spectrum of the photoactive dye.

Additional features and advantages will be set forth in the description that follow and in part will be apparent from the description, or may be learned by practice of the invention. The objects and other advantages will be realized and attained by the devices, formulations and methods disclosed in the written description and claimed by the claims, as well as the appended drawings.

It is to be understood that both the foregoing general description and the following detailed description are exemplary and explanatory and are intended to provide further explanation of the invention as claimed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows a nail stained with the current standard of using 2% MB in water solution.

FIG. 1B shows a nail stained with a formulation which shows a significant diffusion of the dye into the nail plate. This significant enhancement enables efficient treatment of deeply embedded fungi or bacteria.

FIG. 2A shows the right side of the nail stained with a formulation using riboflavin as a photoactive dye vs the left side treated with a standard formulation.

FIG. 2B shows the right side of the nail stained with another formulation using MB as a photoactive dye vs the left side treated with a standard formulation.

FIG. 3 is a functional block diagram of an embodiment of a device or system for the treatment of onychomycosis.

FIG. 4 is a functional block diagram an embodiment of another device or system for the treatment of onychomycosis.

FIG. 5A illustrates an embodiment of the illuminator of the system(s) of FIG. 3 and/or FIG. 4. FIG. 5B illustrates an example of the local intensity distribution of light emitted by an example embodiment of the illuminator of FIG. 5A.

FIG. 6 shows in detail one possible embodiment of the drive and control electronics of the system(s) of FIG. 3 and/or FIG. 4.

FIG. 7 shows an embodiment of an illuminator comprising an array of light sources.

FIG. 8A illustrates a flowchart of an exemplary method in accordance with exemplary embodiments. FIG. 8A illustrates a method in which the nail it treated and evaluated after each treatment to see if retreatment is necessary. FIG. 8B illustrates a flowchart of another exemplary method in accordance with other embodiments. FIG. 8B illustrates is a fixed cycle treatment regimen requiring a fixed N-number of treatments to successfully treat the nail.

FIG. 9 shows a toe nail under exposure using an LED-based illuminator.

FIG. 10 shows a partly stained nail followed for an extended period of time.

### DETAILED DESCRIPTION OF PREFERRED EMBODIMENTS

As used herein, when a quantity, concentration, percentage, or other relationship is said to be “about” a particular value, it means that it is within +/- 10% of that value. So, for example, a concentration of about 20% would be between 18% and 22%. As used herein, a kit refers to a set of articles or equipment which can be utilized together for a particular purpose. The articles in a kit may be manufactured, marketed, or sold together or separately.

Fingernails and toenails are made of keratin, a polymer. The nail plate is produced by the nail matrix which created cells that become the nail plate as they pushed older cells forward. This nail growth is on average about 3.5 mm per month for finger nails and about 1.6 mm per month for toe nails.

Onychomycosis is a fungal infection of the nail, mostly caused by dermatophytes. These fungi affect the nail plate, nail bed and matrix as they require keratin for growth. This infection is rather common and affects about 10% of global population. This infection is difficult to treat due the low ability of most drugs to effectively penetrate the nail - inhibiting an effective treatment. At the same time the low growth rate of the nail, which also further decreases with age, leads to extremely long treatment durations of about a full year to allow the regrowth of a full new unaffected nail. These prolonged treatments require significant patient compliance and adherence over a long period of time. In-office interventions are extremely time consuming and costly due to the duration of the intervention needed.

As disclosed herein, these limitations may be overcome by introducing a new method, device, formulation and kit which enable a highly effective treatment of onychomycosis while showing no side effects and good patient compliance.

The methods and systems disclosed herein provide many advantages over the state of art. Specifically, a highly effective treatment of onychomycosis is enabled through a highly effective penetration formulation of a photoactive dye deep into the nail plate in combination with a very compact and flexible illumination system. In some embodiments, the formulation and the illumination system (including embodiments described below) may be packaged or otherwise provided, marketed, or sold together as a kit or other product. They may also be provided separately and combined into a kit by an end user.

Diffusion of the nail plate is a standard problem for transungual drug delivery. This is also the mayor hurdle for the development of nail lacquers for onychomycosis as it is difficult to get the antifungal drug delivered deep into the affected nail pate in concentrations high enough to be effective. Factors impacting transungual diffusion are molecular size, hydro & lipophilicity, exposure duration, as well as pH and solute charge of the drug and formulation. There are also physical methods to enhance nail penetration which include iontophoresis, etching, micro laser drilling or hydration and occlusion.

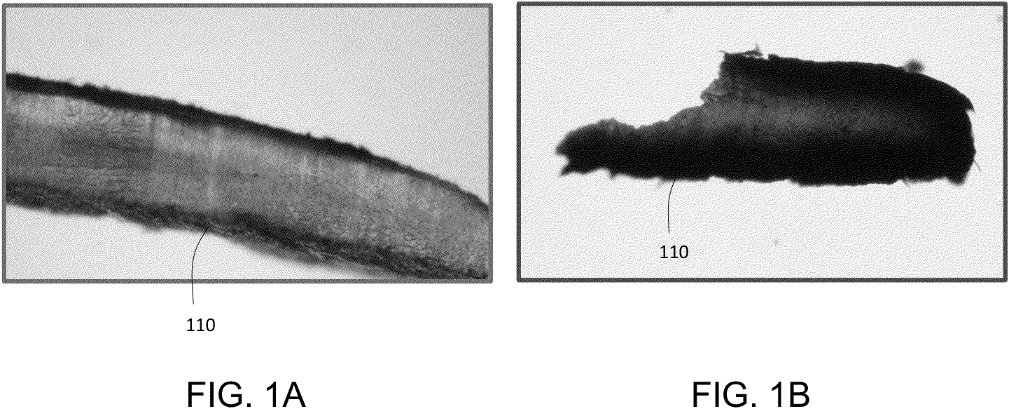

For the photodynamic treatment of onychomycosis, overnight occlusion and hydration was clinically used to potentially soften the nail. This was followed by a short staining cycle of a dye like MB in H2O. FIG. 1A shows a cross section of a nail plate stained with this protocol. In particular, FIG. 1A shows a nail 110 stained with the current standard of using 2% MB in water solution. Only superficial staining is achieved. It is clearly visible that the dye does not penetrate the nail in any significant way.

On the other hand, FIG. 1B shows the cross-section of a nail 110 stained with a new formulation. There is nearly a full penetration of the nail plate with the dye. This significant enhancement enables efficient treatment of deeply embedded fungi or bacteria.

The formulation utilized for extremely high diffusion of MB as shown in FIG. 1B consists of 2% MB and a concentration of urea of about 40%. The formulation utilizes a concentration of urea along with deionized water, oils, emulsifying wax, glycerin, propylene glycol, glyceryl stearate, PEG100, Cetyl Alcohol, alkyl benonate, pheoxylethanol, and various paraben contents. In some embodiments, the concentration of urea is between 0.1% and 30%. In some embodiments, the concentration of urea is about 20%.

This formulation is applied directly to the nail and utilizes a long penetration duration of 6-12 hours. Covering the area during this period is essential for several reasons. First, the formulation should not dry out during the staining process as its moisture and increased water content of the nail plate allows the deep integration and penetration of the dye. Second, the cover prevents the stain from spreading to unwanted areas. Possible solutions can range from regular tapes and drapes, to more advanced coverings which include depot areas within the cover to store extra formulation. However, a full finger or toe cover is also possible.

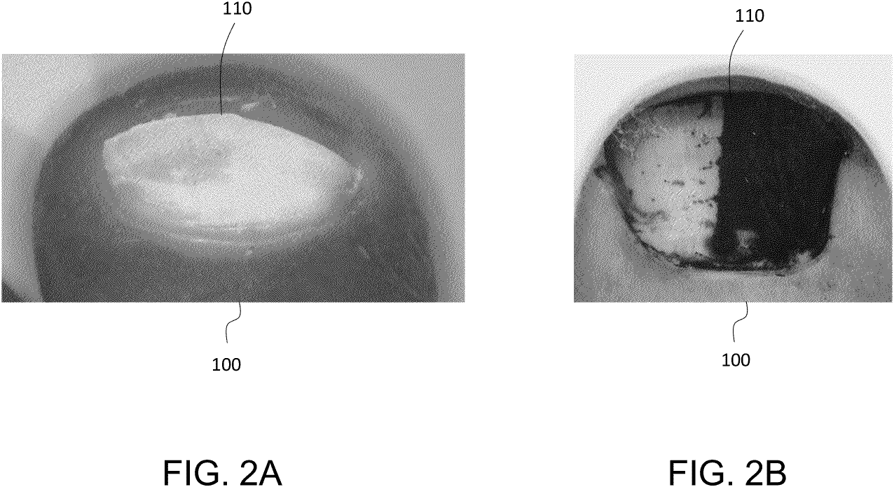

It is important to point out that this new formulation not only works with one specific stain but generally applies to a wide range of possible photodynamic stains. FIG. 2A shows the successful deep stain of the same formulation using riboflavin (RF) in a half nail experiment. In this experiment the right side of a nail was stained with the new formulation and RF while the left side of the same nail was stained with the standard technique which results in nearly no uptake of the dye within the nail plate. The image was takes by exposing the nail additionally to UV light to excite the yellow fluorescence of the RF dye. Under just visible light the stain of RF is not very visible.

The tremendous gain of the dye concentration within the nail plate of this new formulation is also very visible in the direct comparison with the standard stain method on the same nail.

FIG. 2A shows the right side of a nail 100 stained with a formulation using riboflavin as a photoactive dye vs the left side treated with a standard formulation. The fluorescence shows a deep uptake of the dye on the right side while nearly no uptake for the standard formulation.

FIG. 2B shows the right side of the nail 100 stained with another formulation using MB as a photoactive dye vs the left side treated with a standard formulation. The stain profile shows a deep uptake of the dye on the right side while nearly no uptake happened for the standard formulation.

It is also important to point out that with this novel formulation the dye is deeply integrated into the nail plate and does not diffuse or leak out over a short period of time. To demonstrate that, the nail of FIG. 2B (stained with MB in the novel formulation) was followed for a long time period - 6 months.

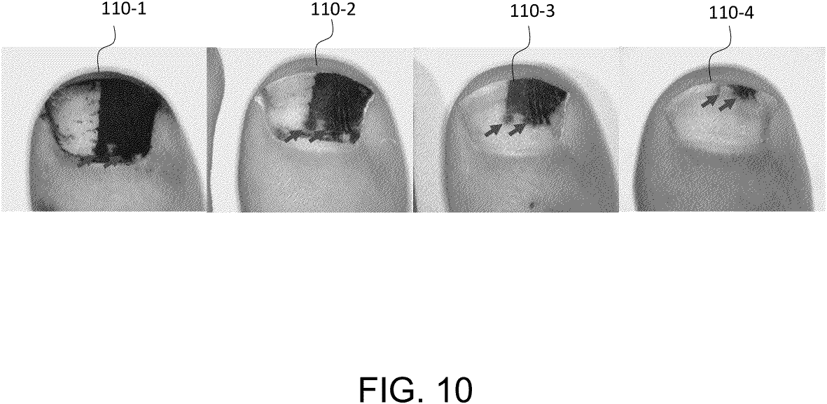

FIG. 10 shows that the stain is maintained over this period while the nail grows out. In FIG. 10, 110-1 is the nail after one day, 110-2 is the nail after one month, 110-3 is the nail after two months, and 110-4 is the nail after six months - the full growth period for this specific nail. No reduction in contrast, which would indicate a washout, is visible. The small arrows in FIG. 10 show the local contrast variabilities which maintain over time as the nail grows out completely. This demonstrates full and deep integration of the dye into the nail plate as it stays until the nail grows out. The novel formulation allows deep penetration of the dye into the nail plate in which it is trapped allowing a long and sustained ability to treat the infected nail with the photochemical activation of singlet oxygen through activation with a spectrally matched light source.

Beneficially, dye concentration in the novel formulation ranges from 0.01% to 10%; more specifically it is preferred that it be from about 0.1% to about 5%. The final concentration depends on the linear absorption created within the nail plate due to the diffusion of the dye. A concentration which is too high is not advisable as all the light will get absorbed within the superficial layer of the nail plate, while a concentration which is too low might allow good light penetration but might be too low in concentration to effectively treat the underlying root cause through photoactivation. The preferred concentration is configured to allow an effective treatment while still allowing deep light penetration into the stained nail bed. However, this also depends on the wavelength utilized as UV light and blue wavelength light have difficulty penetrating into the nail due to increased light scattering. Light in the red or near IR wavelengths is preferred in that sense, but also here a balance needs to be taken into account between high quantum yield which typically happens with higher energetic UV or blue light photons and the good light penetration of longer wavelengths.

Good dye candidates but not limited are MB and RF but also indocyanine green (ICG). Other possible candidates are Verteporfin, Redaporfin, Padeliporfin, as well as Zinc- or Aluminum Phthalocyanine Tetrasulfonates.

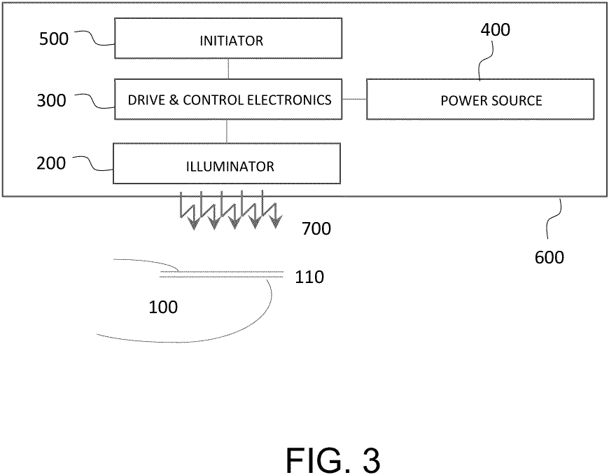

FIG. 3 is a functional block diagram of an embodiment of a device or system 600 for the treatment of onychomycosis. System 600 comprises an illuminator 200, drive and control electronics 300, a power source 400, and an initiator 500, such as a trigger, pushbutton, or the like. Illuminator 200 is operatively coupled to drive and control electronics 300. Drive and control electronics 300 is supplied by power from power source 400. In some embodiments, power source 400 may comprise one or more batteries, which may be rechargeable. Drive and control electronics 300 may be triggered to start by initiator 500 and may drive and control illuminator 200 to emit light 700. The emitted light from system 600 may be used to illuminate a toe or finger 100 with a nail 110 which is stained with a photoactive dye. Beneficially, emitted light 700 of illuminator 200 is spectrally matched to the activation and absorption of the photoactive dye used to stain nail 110. Beneficially, a spectral peak of emitted light 700 from illuminator 200 is within 50 nm of a targeted activation peak of the photoactive dye. More beneficially, the spectral peak is within 10 nm of the targeted activation peak of the photoactive dye.

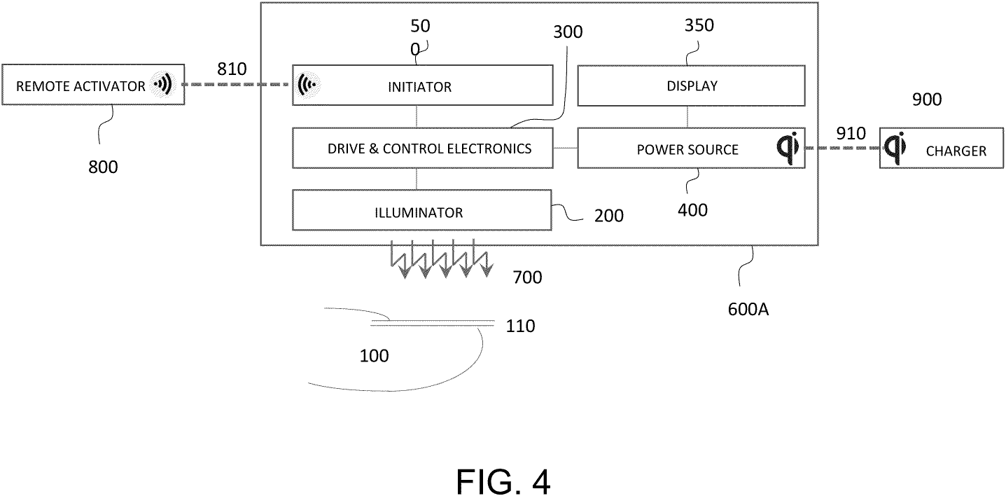

FIG. 4 is a functional block diagram of an embodiment of another device or system, 600A, for the treatment of onychomycosis with enhanced features compared to system 600. In particular, system 600A includes a display 350, an external device 800 such as a remote activator, a connection 810, a charger 900 and a connection 910. External device 800 can be a separate device such as a mobile phone with a connected application or a dedicated device. Also data can be transmitted through connection 810 from and to external device 800. In various embodiments the communication between external device 800 and initiator 500 can be wireless through Bluetooth, near-field communication (NFC) and/or WiFi. Charging the battery or rechargeable battery 400 can be done with a wired or wireless connection 910 to the charger 900. In one possible embodiment, wireless charging established standards like Qi can be implemented. Also, display 350 may be utilized to allow interaction with the user and possibly display information about the device status and or treatment progress.

Besides the novel formulation of allowing a deeper and long lasting penetration of the photoactive dye into the nail plate, the system 600, which activates the dye, may also novel features. Illuminator 200 of systems 600 and 600A as illustrated in FIGS. 3 and 4 provides a light emission that has significant spectral overlap to the activation spectrum of the photoactive dye in use. Its light output is high enough so an effective treatment can be enabled. Typical irradiances range from 0.0001-10 W/cm2, specifically 0.003-0.2 W/cm2.

Candidates for light sources include light emitting diodes (LEDs), diode lasers, vertical cavity surface emitting lasers (VECSELs) or VECSEL-arrays. LEDs have the advantage that they are now powerful enough to deliver these irradiances at a low cost point but come with a broader light spectrum. Diode lasers or VECSLs are higher in price but are able to specifically target the peak absorption wavelengths of the photoactive dye in use.

Beneficially, these light sources are arranged so that they can emit a uniform light distribution at the target. This allows a uniform activation of the dye.

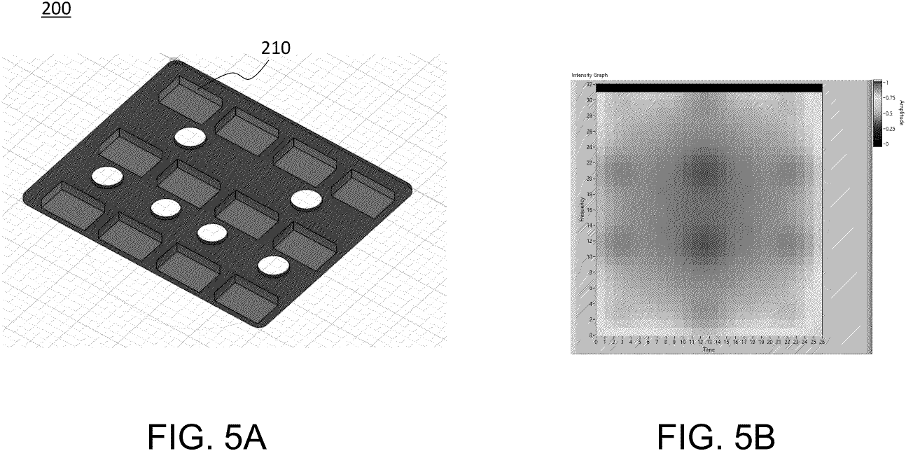

FIG. 5A illustrates an example embodiment of illuminator 200, which may be operationally coupled to drive and control electronics 300. Beneficially, illuminator 200 may comprise an array of spectrally matched light sources 210 such as LEDs. Here, the distances between light sources 210 in the array may be carefully chosen so that the irradiance levels at the correct working distance to the nail to be treated are within 15-20% of peak intensity. FIG. 5B illustrates an example optical simulation of the local intensity distribution of light emitted by an example embodiment of the illuminator of FIG. 5A. Here, the local intensity distribution is between 80% and 100% within the field of interest.

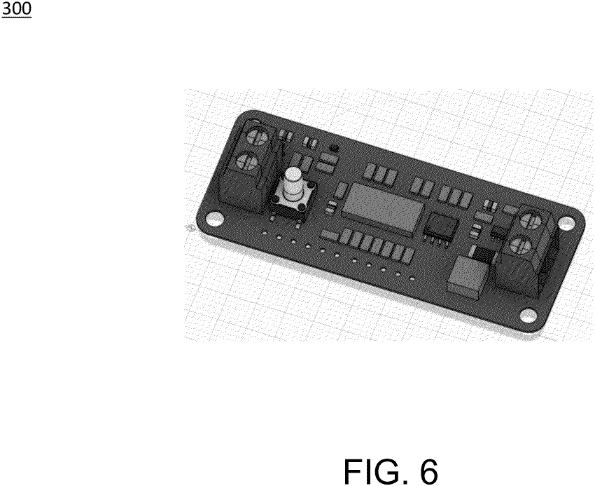

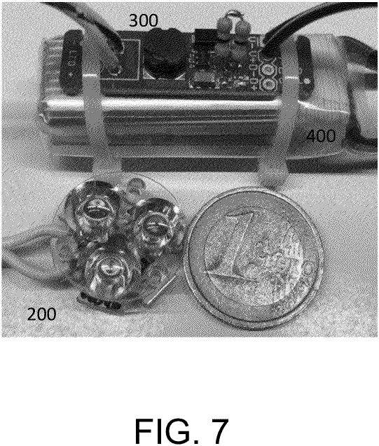

Instead of, or in addition to the array of FIG. 6, beam forming optics can be utilized. as seen in the example of illuminator 200 of FIG. 7 in which an array of 3 LEDs are operatively coupled with a refractive and total reflection beam forming optics. Additional options to create a uniform light intensity include light diffusors or structured transmissive or reflective surfaces to homogenize an otherwise inhomogeneous light beam.

Illuminator 200 is driven and controlled by dedicated driver and control electronics 300, which is configured to enable a correct activation as well as timing of the light exposure and the control of the light intensity output. Beneficially, driver and control electronics 300 is implemented by an electronics board which is operatively coupled to illuminator 200. Beneficially, driver and control electronics 300 includes a timer to control on - off timing of illuminator 200. The timer may comprise, for example, a small microcontroller (e.g., a PIC16F15244) or a timing module like a TI-555 timer. The on duration for a single exposure setting can be in the range of 3 sec to 3 hours, but is typically 5-30 minutes. Beneficially, driver and control electronics 300 includes a brightness control mechanism for controlling an intensity of light 700 emitted by illuminator 200, which in some embodiments may be adjusted under user control, for example by a control knob, a slide mechanism, or the like. In some embodiments, the brightness control mechanism includes a voltage-to-current converter configured to supply current to illuminator 200. In some embodiments, the brightness control mechanism includes a pulse with modulator for applying pulse width modulation (PWM) to the drive current supplied to illuminator 200.

Driver and control electronics 300 can also serve other additional functions, such as driving an IO system such as display 350 as shown in FIG. 4. Additional functions might include wireless communication with an external device 800, such as a mobile phone or other device, battery charge state and charge controller, proximity and temperature sensors. External interfacing and communication can be achieved by direct I/O, analog-to-digital conversion (ADC) or digital-to-analog conversion (DAC) communication or through the addition of additional communication ports, such as I2C. This all can be handled through a small microcontroller, such as the PIC16F15244 mentioned above. However, many other options are available for a task like that. Driver and control electronics 300 can also be operatively coupled to a Bluetooth or NFC module for communication with external device 800, which can be a cell phone or other device. External device 800 can contain an application (app) which stores and records treatment data, tracks progress and also reminds the patient about future treatment sessions. External device 800 can be used to take pictures of the affected nails and analyze these data via direct surgeon feedback, AI analysis or a reading center. This help and support system allows the patient to optimally treat the affected area. The gathered data can also be used to further optimize the treatment algorithm and settings as well as do deep data analysis for other applications.

FIG. 6 shows an example of such drive and control electronics 300 with an included microcontroller, current and voltage converters (buck boost) as well as the connection to battery 400 and illuminator 200. Initiator 500 is already integrated on this board for simplicity. Drive and control electronics may be used to control the output of illuminator 200, interface with display 350, and/or interface through connection 810 with external device 800 such as a remote activator. In this embodiment a micro controller is included which may allow full control of the light intensity and duration of light 700 emitted by illuminator 200.

Driver and control electronics 300 is powered through battery 400, which may be a rechargeable battery like a LiPo battery. The use of a battery or rechargeable battery also allows the full mobility of the device to be used wherever the user decides to perform treatment. With battery 400, no power supply or cables are needed during operation. As indicated in FIG. 4 the battery can also be charged wirelessly through inductive charging, such as provided by like the Qi standard, or a wired connection. Charger 900 is the matching power supply.

The electronics can be activated through initiator 500 which is operatively coupled to drive and control electronics 300. This allows the user to activate system 600, and in particular illuminator 200.

Overall the system 600 is encased to allow the illumination of the nail 110 of a finger or toe 100. This includes mechanical means to block parts of the light 700 from exiting the system other than the aperture used for illuminate the nail plate 110. This allows an overall light-safe operation of the system 600 which emits quite significant amounts of light which need to be mitigated to be eye-safe for example. This can be achieved through a base plate underneath the finger or toe 100, and other light limiting shields.

FIG. 7 shows another example of a simpler embodiment of drive and control electronics 300 along with battery 400 and illuminator 200. In this example the LED output power is controlled through a dedicated drive constant current controller using a simple buck-boost converter and a manual on off switch.

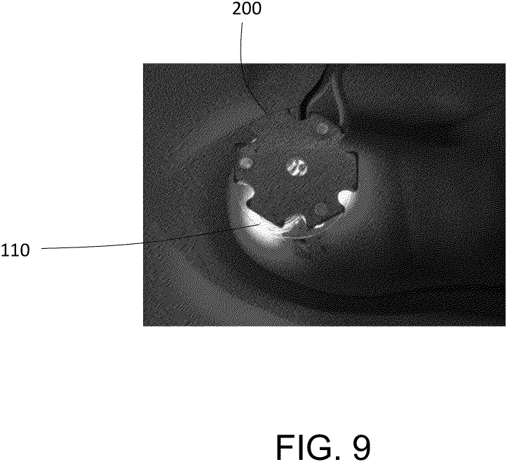

FIG. 9 shows the application of the illuminator 200 on a stained nail plate 110. In this embodiment, the LED array and focusing optics are mounted on an aluminum PCB board to better control heat diffusion from the LEDs and allow a constant output power of the array under constant current drive conditions. Even with this focusing optics a lot of light is still escaping from the device making it clear how important an encapsulation is to make sure light levels are preserved for everyday consumer usage.

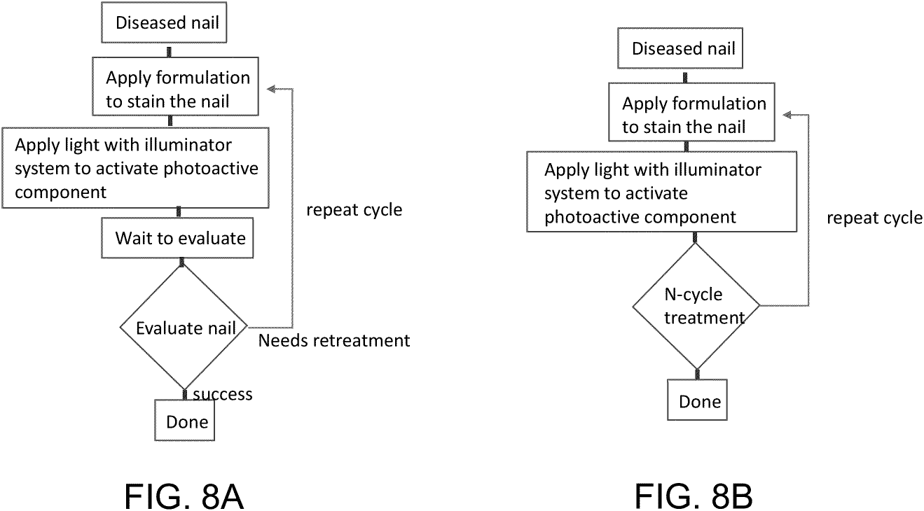

Different usage flow charts and scenarios may be employed. As depicted in FIG. 8A, one embodiment includes staining the affected nail and waiting for it to penetrate deep into the nail plate, then applying light with an illuminator system (e.g., system 600 or 600A) and wait a certain time to evaluate the outcome. Based on the evaluation the nail is retreated or the treatment cycle is finished. This evaluation can be through the patient itself but also through photos taken with an independent device like a cell phone or a linked device such as remote device 800 and sent for remote analysis. This remote analysis could be AI driven or by a remote clinical team. This dynamic treatment regimen may allow the fastest possible treatment with the lowest number of cycles due to the included feedback cycle. Cycle times are typically within 1-2 weeks time frame but can stretch out to 1-3 months.

In another embodiment, the treatment cycle runs through a fixed number of cycles as depicted in FIG. 8B. In this embodiment, the affected nail is stained, followed by the light activation of the dye with the illumination system. Here, this cycle is repeated a fixed number of times, N, ensuring thorough treatment of the nail bed. Cycle times are typically within 1-2 weeks time frame but can stretch out to 1-3 months. N may be any appropriate number, for example 10.

The various components and modules of the system described above may be implemented by electrical circuitry including logic circuits, and/or processors which execute computer executable program code stored in computer readable non-volatile memories and other tangible, non-volatile media.

It will be apparent to those skilled in the art that various modification and variations can be made in the methods, system, and formulations disclosed herein. Thus, it is intended that the present invention be defined by the appended claims, including the recited elements and their equivalents.

What is claimed is:

1. A kit for treating an infection of a nail, the kit comprising:
a formulation including urea, and a photoactive dye to stain the nail; and
a device for illuminating the stained nail, wherein the device includes:
an illuminator for emitting light to the stained nail having the formulation applied thereto;
drive and control electronics for driving and controlling the illuminator;
an initiator for commencing illumination of the stained nail having the formulation applied thereto; and
a power source for supplying power to the drive and control electronics and the illuminator.

2. The kit of claim 1, wherein the formulation is in one of a tube, a jar, a roll-on applicator, an applicator strip or a prefilled brush pen.

3. The kit of claim 1, wherein the illuminator includes an array of light sources.

4. The kit of claim 3, wherein the light sources include light emitting diodes (LEDs).

5. The kit of claim 1, wherein the photoactive dye has an absorption spectrum, and the emitted light of the illuminator is spectrally matched to the absorption spectrum of the photoactive dye.

6. The kit of claim 1, wherein the photoactive dye includes at least one of Methylene Blue, Riboflavin-5-phosphate and Riboflavin.

7. The kit of claim 1, wherein a concentration of the photoactive dye in the formulation is between 0.001% and 10%.

8. The kit of claim 1, wherein a concentration of the photoactive dye in the formulation is between 0.1% and 2%.

9. The kit of claim 1, wherein a concentration of the urea in the formulation is between 0.1% and 30%.

10. The kit of claim 1, wherein a concentration of the urea in the formulation is about 20%.

11. The kit of claim 1, wherein the nail is irradiated by the illuminator with a light irradiance level in a range of 0.001 W/cm2 to 10 W/cm2.

12. The kit of claim 1, wherein the nail is irradiated by the illuminator with a light irradiance level in a range of 0.01 W/cm2 to 0.5 W/cm2.

13. The kit of claim 1, wherein a spectral peak of the light emitted by the illuminator is within 50 nm of a targeted absorption peak of the photoactive dye.

14. The kit of claim 1, wherein a spectral peak of the light emitted by the illuminator is within 10 nm of a targeted absorption peak of the photoactive dye.

15. The kit of claim 1, wherein the drive and control electronics includes a timer configured to control an on-time of the illuminator.

16. The kit of claim 15, wherein the on-time is adjustable by the timer to be a selected value in a range of between 3 seconds and 3 hours.

17. The kit of claim 15, wherein the on-time is adjustable by the timer to be a selected value in a range of between 5 minutes and 30 minutes.

18. The kit of claim 1, wherein the drive and control electronics includes a brightness control mechanism for controlling an intensity of the light emitted by the illuminator.

19. The kit of claim 18, wherein the brightness control mechanism includes a pulse width modulator.

20. The kit of claim 18, wherein the brightness control mechanism includes a voltage to current converter which is configured to control a current which is supplied to the illuminator.

21. The kit of claim 18, wherein the power source comprises a battery.

22. A method of treating an infection of a nail, the method comprising:
applying a formulation to the nail, wherein the formulation includes urea, and a photoactive dye to stain the nail; and
illuminating the stained nail having the formulation applied thereto with light emitted from a device which includes a light generator,
wherein the emitted light includes wavelengths within an absorption spectrum of the photoactive dye.

23. The method of claim 22, wherein artificial light generator includes an array of light sources.

24. The method of claim 23, wherein the light sources include light emitting diodes (LEDs).

25. The method of claim 22, wherein the photoactive dye has an absorption spectrum, and the emitted light is spectrally matched to the absorption spectrum of the photoactive dye.

26. The method of claim 22, wherein the photoactive dye includes at least one of Methylene Blue, Riboflavin-5-phosphate and Riboflavin.

27. The kit of claim 1, wherein a concentration of the photoactive dye in the formulation is between 0.001% and 10%.

28. The method of claim 22, wherein a concentration of the photoactive dye in the formulation is between 0.1% and 2%.

29. The method of claim 22, wherein a concentration of the urea in the formulation is between 0.1% and 30%.

30. The method of claim 22, wherein a concentration of the urea in the formulation is about 20%.

31. The method of claim 22, wherein the nail is irradiated with a light irradiance level in a range of 0.001 W/cm2 to 10 W/cm2.

32. The method of claim 22, wherein the nail is irradiated with a light irradiance level in a range of 0.01 W/cm2 to 0.5 W/cm2.

33. The method of claim 22, wherein a spectral peak of the emitted light is within 50 nm of a targeted absorption peak of the photoactive dye.

34. The method of claim 22, wherein a spectral peak of the emitted light is within 10 nm of a targeted absorption peak of the photoactive dye.
